(19) United States
(12) Patent Application Publication
(10) Pub. No.: US 2025/0384703 A1
(43) Pub. Date: Dec. 18, 2025

(54) Method for repositioning a focus position of an imaging device into a target focus position

(71) Applicant: Carl Zeiss Microscopy GmbH, Jena (DE)

(72) Inventors: Manuel Amthor, Jena (DE); Daniel Haase, Zoellnitz (DE); Thomas Ohrt, Golmsdorf (DE)

(21) Appl. No.: 19/229,654

(22) Filed: Jun. 5, 2025

(30) Foreign Application Priority Data
Jun. 17, 2024 (DE) 10 2024 116 985.0

(51) Int. Cl. G06V 20/69; G01N 33/483; G06V 10/26; G06V 10/74; G06V 10/766; G06V 10/96; G06V 10/98; H04N 23/61; H04N 23/67

(52) U.S. Cl. CPC G06V 20/693; G01N 33/4833; G06V 10/273; G06V 10/761; G06V 10/766; G06V 10/96; G06V 10/993; G06V 20/695; G06V 20/698; H04N 23/61; H04N 23/67

(57) ABSTRACT

A method for repositioning a focus position of an imaging device in a target focus position in a sample in an experiment comprises: defining the target focus position, repositioning a current focus position based on the target focus position, comprising determining one or more compare signatures based on a current focus position, determining one or more distances in each case between the compare signatures and a target signature based on the target focus position, adapting the current focus position based on the distances. A signature is an output of a machine learning model corresponding to a focus position and based on an image of the sample recorded with the focus position, and the target focus position is a focus position in the sample in which the imaging device captures a target image of the sample and the machine learning model outputs the target signature when the target image is input.

### RELATED APPLICATIONS

This application claims priority to German Patent Application No. 10 2024 116 985.0, filed on Jun. 17, 2024, which is incorporated herein by reference in its entirety.

### BACKGROUND OF THE INVENTION

In the prior art, time series of samples comprising a plurality of images are recorded by imaging devices, in particular microscopes, wherein a focus position of the imaging device in the sample remains as constant as possible over the images of the time series, wherein constant in this sense means that the same sample structure is always captured in focus by the imaging device in the images of the time series. Three technological possibilities for stabilizing the focus position of an imaging device are known from the prior art: by increasing the mechanical stability of the imaging device used, by using a hardware-based holding focus (definite focus) or by using a software-based holding focus, wherein, for example, the software-based holding focus determines a sharpest, most intense or most contrast focus position, in particular by means of a sharpness measure, an intensity measure and/or a contrast achieved during the imaging.

An increased mechanical stability is achieved for imaging devices, in particular microscopes, by stiffening the microscope including the sample or including the connection to the sample. A stiffening of the microscope increases the weight and the costs, and furthermore reduces the flexibility with respect to the samples that can be used, since access to the sample holder is made more difficult by the stiffening or the connection of the microscope to the sample holder or the sample, in particular on account of the geometric restrictions. However, such mechanically stabilized microscopes do not prevent drifting of the focus position, caused, for example, by expansion or contraction of the components of the microscope triggered by fluctuating ambient temperatures, for example caused by switching a light source of the microscope on and off during an experiment.

A hardware-based holding focus is available for certain immersions, for example air, water or silicone oil. For the use of a hardware-based holding focus, the microscope requires an additional module, such as, for example, the definite focus module of the applicant. In the case of the hardware-based holding focus, for example, a grating with an infrared light source is projected onto a reflective surface of the sample holder, the grating is imaged on the image sensor, and a displacement of the grating on the image sensor corresponds to a drift of the focus position. By correspondingly displacing the focus position, the grating projection can be displaced back into its original position again. This original position corresponds precisely to the desired focus position. In the case of microscopes with a hardware-based holding focus, problems can arise, for example, as a result of deviations from cover glass parameters, such as, for example, thickness or parallelism, if, for example, relatively large samples are observed. Furthermore, the hardware-based holding focus does not function for samples which are freely movable in a solution, since their position in a direction perpendicular to the illumination can change over the duration of the experiment. Furthermore, there are samples in which structures grow perpendicularly to the reflective surface. The hardware-based holding focus also does not function for such samples. Therefore, the hardware-based holding focus also has certain restrictions with respect to its applicability to certain samples.

In the case of microscopes with a software-based holding focus, the software-based holding focus determines, for example, a sharpness measure for different focus positions of the microscope along the depth of the observed sample, in each case for each of the recorded focus positions, and selects the focus position with the highest sharpness as the focus position to be held. This software-based holding focus functions well for immutable samples and for thin samples, i.e. the thickness of the sample lies within an objective depth of field of the microscopy system. However, if the microscopy system observes samples with a thickness greater than the objective depth of field of the microscope or if the sample changes over time, the robustness and the reproducibility of the software-based holding focus are reduced. If the samples to be imaged contain, for example, organisms whose development is to be examined, the changes with respect to the focus position in the sample are so great that a software-based holding focus can no longer be used. Furthermore, it may be desirable to observe small regions in samples, such as, for example, a lower polar region of an embryo. This would always have a lower value with respect to the sharpness measure in comparison to an equatorial plane of the embryo, and therefore the software-based holding focus could not automatically focus the lower polar region.

Precisely in recent years, it has been observed that more and more users of microscopes are examining thick samples. Depending on the sample, i.e., for example, depending on an immersion used, observation is only possible with the aid of a very expensive hardware-based holding focus. The hardware-based holding focus is used only with certain immersions, in particular air, water and silicone oil, and furthermore deviations from cover glass parameters, such as thickness and parallelism, lead to problems, in particular in the case of relatively large samples. Furthermore, the hardware-based holding focus does not function in the case of samples which are freely movable in the respective solution or which grow asymmetrically. For samples for which the hardware-based holding focus is unsuitable, only a software-based holding focus or the increase in the mechanical stability of the microscope remains. However, these solutions still do not permit reliable observation in a time series of a certain sample structure selected by the user, since they do not permit reliable focusing of arbitrary focus positions in which the selected sample structures are correspondingly well imaged.

The methods known from the prior art for automatically focusing a microscope within a sample, despite high investments in new hardware, do not exhibit sufficient stability and robustness with respect to automatic focusing of a desired focus position, in particular in the case of thick and/or time-variable samples.

### SUMMARY OF THE INVENTION

There is therefore a need to improve the robustness and quality with which a focus position in a sample is automatically held, and to reduce the costs for such a microscope. The present invention relates to a method for repositioning a focus position of an imaging device and to an evaluation device and to a repositioning system comprising the evaluation device for carrying out the method and to a computer program product.

It is the object of the invention to provide a method in which a microscope can focus as automatically as possible on a certain structure within a sample or a focus position of the microscope in the sample can be held on a desired sample structure, the target sample structure, without causing additional costs and without there being restrictions with respect to the type of sample used.

One or more objects are achieved by the subject matter of the independent claims. Advantageous developments and preferred embodiments form the subject matter of the dependent claims.

One aspect of the invention relates to a method for repositioning a focus position of an imaging device into a target focus position of an experiment. The method comprises the steps of
defining the target focus position,
repositioning a current focus position based on the target focus position, wherein the repositioning of the target focus position comprises:
   determining one or more compare signatures based on a current focus position,
   determining one or more signature distances in each case between the compare signatures and a target signature based on the target focus position,
   adapting the current focus position based on the signature distances,
      characterized in that a signature is an output of a machine learning model corresponding to a focus position and based on an image of a sample structure recorded with the focus position, and the target focus position is a focus position in the sample in which a target imaging device captures a target image of the sample and the machine learning model outputs the target signature when the target image is input, wherein the target image in particular captures a target sample structure.

According to some embodiments of the present invention, a focus position of an imaging device in a sample is the region in the sample which the imaging device images with a maximum sharpness. The focus position of an imaging device is variable; for example, a sample holder or an imaging device can be displaceable along an axis, this axis is usually referred to as the Z axis; by displacing the sample along the Z axis, the focus position of the imaging device is displaced within the sample. Alternatively, an optical system, comprising for example at least one objective, can also be displaceable along the Z axis, while the sample holder is not displaceable. Alternatively, the sample holder and the optical system can also be displaceable.

According to some embodiments of the present invention, a signature is an output of a machine learning model, in particular an output of one or more intermediate layers of a machine learning model, which is output when an image is input into the machine learning model. The signature is present in particular as a vector or general tensor. The signature accordingly represents a point in a high-dimensional feature space; the feature space is also called embedding space.

According to some embodiments of the present invention, a sample structure is a structure contained in a sample which can be recorded in particular with the imaging device. Sample structures can in particular be extended objects which can be captured with different focus positions with the imaging device.

According to some embodiments of the present invention, a target sample structure is precisely the structure which is to be examined in an experiment. In particular, the target sample structure is to be examined in the target focus position. Some sample structures are so-called flat structures; these are imaged sharply by the imaging device only in a single focus position. Furthermore, some samples also have thick structures. For thick structures, different parts of the structure are captured in focus at different focus positions of the imaging device in the sample. If a thick structure is to be captured as the target sample structure via the experiment, the target focus position corresponds precisely to the structures or regions of the target sample structure which are imaged sharply in the target focus position.

According to some embodiments of the present invention, the expression “repeatedly comprising” means that the respective steps are carried out multiple times in succession.

In the following, the expression experiment means a repeated recording of a sample with an imaging device, wherein the sample is or can be variable in particular over the course of the experiment.

According to some embodiments of the present invention, samples can be any desired objects, fluids or structures, for example biological structures. Each sample is suitably arranged and fixed in the beam path of an imaging device by means of a sample carrier.

According to some embodiments of the present invention, a machine learning model, in particular a neural network, is a processing model which can be trained in particular by means of a supervised or unsupervised learning process for processing input data and outputting output data. In particular, randomly initialized machine learning models can be used, as can pre-trained models or also fully trained models.

According to some embodiments of the present invention, the processing by means of a processing model comprises inputting an input datum or a plurality of input data into the processing model and outputting an output datum by the processing model.

According to some embodiments of the present invention, the input data can be in particular images, image stacks and time series of images or image stacks.

In particular, according to some embodiments of the present invention, in addition to the output datum, an intermediate output datum of an intermediate layer of the machine learning model can also be used, wherein an intermediate layer is a layer of the processing model, the output datum of which is used as input datum in a following layer in the machine learning model.

The processing of images according to some embodiments of the present invention can comprise a wide variety of processing images.

Whether the machine learning models are randomly initialized models, pre-trained models or fully trained models results in each case from the context or is explained accordingly.

According to some embodiments of the present invention, the training of a machine learning model is understood to mean a supervised learning or an unsupervised learning, in particular a self-supervised learning.

In the supervised learning, an annotated data record is used. The annotated data record comprises input data and target data, wherein an annotation or identification, called target datum, of the target data corresponds to each input datum of the input data.

The target datum is a datum used in the training of the processing model for carrying out a processing image, to which an output datum output by the processing model on the basis of the input datum is to be adapted. The approximation is carried out with the aid of an objective function. The objective function is, in particular, a gain or loss function which specifies how distances, differences or else a degree of correspondence between the output datum of the processing model and the target datum are evaluated. The evaluation can be carried out entry by entry on the basis of the entries of the respective data or by a comparison of more abstract entities.

The loss function can capture, for example, differences between the output datum and the specified target datum. If the input datum and the target datum are images, for example, the comparison can be carried out pixel by pixel. The pixel-by-pixel differences can be added in absolute value (as absolute values) in an L1 loss function. The square sum of the pixel-by-pixel differences is formed in an L2 loss function. In order to minimize the loss function, the values of model parameters of the processing model are changed, which can be calculated, for example, by gradient descent and back propagation.

In the unsupervised learning, as is used for training in autoencoders, for example, the training data comprise only the input data, but no target data, or the input data are also the target data at the same time.

According to some embodiments of the present invention, a target imaging device is an imaging device with which a target image of the sample was captured. The target imaging device can be the imaging device which is used in a current experiment and which has recorded the target image in the course of the experiment or in a previous experiment with a sample of the same sample type. The target imaging device can also be another imaging device, for example also an imaging device which has recorded the target image with a different image contrast or with a different imaging, while the respectively different imaging is used in the experiment. For example, different imaging devices can comprise a bright field microscope, a dark field microscope, a light sheet microscope, laser scanning microscope or also a multiphoton microscope. For example, other fluorophores, other exposures or also other filters can also be used in the recording of the target image than in the experiment.

In conventional methods for repositioning a focus position, hardware-based approaches, software-based approaches or approaches for the mechanical stabilization of an imaging device are used as described above. All these approaches have different disadvantages; in particular, it is not possible with any of the described approaches to hold a focus position for any of the different sample structures arranged in different focus positions in a thick sample, that is to say a sample with a plurality of planes of interest for an experiment with different sample structures, precisely in a target focus position over the course of an experiment, in which target focus position the selected sample structure is captured in the desired manner, or to reposition the focus position of the imaging device such that it is always in the target focus position as accurately as possible.

The inventors of the present invention have recognized that a machine learning model can be set up or trained such that it outputs, for example in one or more intermediate layers, a signature which is characteristic of the sample structure captured with the imaging device. Based on a target signature which is based on an image recorded in a target focus position of a target sample structure, the machine learning model can thus be used to determine whether the focus position of the imaging device is still in the target focus position and to correct a focus position accordingly by determining one or more compare signatures. The present invention thus provides a method with which an imaging device for repositioning a focus position into a target focus position can be controlled on arbitrarily selected target sample structures.

Preferably, the defining of the target focus position comprises recording the target image and outputting the target signature, based on the target image.

By recording the target image before the output of the target signature, a quality of the target signature can be improved.

Preferably, the recording of the target image comprises recording one or more images having height-offset focus positions, respectively, comprising the target image, and selecting the target image from the plurality of images, whereby the target image can be selected in a particularly simple manner as the image with the best focus position or with the target focus position.

In particular, the recording of the target image is carried out with the imaging device or with the target imaging device.

Preferably, the method also comprises inputting the plurality of images into the machine learning model and identifying the target image, based on the target signature.

For example, a user can select the target focus position or the target image with the target sample structure, respectively, based on a previous experiment. In a new experiment with a sample of the same sample type, the user selects the target focus plane based on the target image recorded in the previous experiment. Thereupon, the imaging device records a plurality of images with mutually height-offset focus positions, can recognize the target image based on the target signature and thus control the imaging device into the target focus position, whereby the present invention makes it possible to keep an imaging device in a target focus position corresponding to the target image over a plurality of experiments.

Images with mutually height-offset focus positions comprise in the following images whose image section at least partially coincides, so that the coinciding image sections can be registered with one another. The focus positions having height-offset to one another have a distance to one another, which in the following is also called focus position distance or height offset.

In particular, a target sample structure is captured in focus by the imaging device in the target image. In particular, the target sample structure in the target focus position is captured sharper than the other sample structures in relation to other sample structures in the respective sample. In particular, the target focus position is not necessarily the focus position at a height of the sample which has a maximum sharpness over the entire image.

Preferably, the selecting of the target image comprises
inputting the plurality of images into the machine learning model, wherein the machine learning model is embodied as a candidate extraction model,
outputting a plurality of candidate images by the candidate extraction model such that images from the plurality of images that do not capture candidate sample structures are filtered out by the candidate extraction model, and
selecting the target image from the candidate images,
   wherein candidate sample structures are captured in the candidate images, in particular the candidate sample structures are biological structures of interest in the sample, in particular one or more of cell edge, cell organelle, cell nucleus, cytoskeleton or mitochondria.

Due to the fact that the machine learning model was trained to first output a selection of candidate images having sample structures of interest, the target sample structures in the candidate images can be identified and correspondingly selected in a simple manner.

Preferably, the machine learning model has been trained over the course of a plurality of experiments, wherein the machine learning model has been trained based on target images selected in the course of the plurality of experiments for outputting the candidate images and the candidate extraction model recognizes sample structures represented in the target images in particular with a high image sharpness.

Due to the fact that the machine learning model is used over a plurality of experiments, for example, a randomly initialized machine learning model can be trained in the course of a plurality of experiments to output candidate images. In particular, the candidate extraction model recognizes the sample structures in the target images which have a particularly high image sharpness. If, therefore, target images of the target sample structures are repeatedly selected by a user, for example, over the course of the plurality of experiments, the candidate extraction model can recognize the respective sample structures in the respective target image, for example, these can be the structures with a highest image sharpness in each case in the target image, i.e. the candidate extraction model learns due to the fact that a user repeatedly selects the target images to recognize precisely the focus position based on the imaged structures.

According to one embodiment, one or more of the following can be variable over the course of an experiment over time: the target sample structure, the target focus position and the target signature.

Due to the fact that the target signature, the target sample structure and the target focus position can be variable over time, the variability also has to be taken into account in each case when repositioning the focus position of the imaging device, if this takes place accordingly, the focus position of the imaging device can also be repositioned accordingly in the case of samples which are variable over time.

Preferably, the target image or the target signature is contained in a sample structure atlas. The sample structure atlas comprises atlas images or atlas signatures or both for biological structures of interest occurring in a sample of a specific sample type, which in particular represent the change over time of the sample. As a result, a signature can be determined, selected or established by the machine learning model for any desired of the relevant or interesting biological structures in the sample based on the sample structure atlas, and the focus position of the imaging device can be repositioned on the structure to be imaged sharply, i.e. the target sample structure, based on the respective signature.

Preferably, the sample structure atlas comprises images of the biological structures of interest or the corresponding signatures determined therefrom in each case for the course of the experiment over time, wherein the sample structure atlas was recorded in particular in one or more previous experiments with a sample of the same sample type, as a result of which the imaging device is also focused on the target sample structures over the course of the experiment over time based on the images of the selected biological structure of interest, i.e. the target sample structures, i.e. the focus position of the imaging device can be repositioned in the corresponding target focus position based on the sample structure atlas.

Preferably, the sample structure atlas comprises focus position information and/or sample position information for each of the atlas images, wherein the focus position information is information with respect to the focus position of the respective sample structure, captured in an atlas image, of the sample structure atlas in the sample with respect to one another, in particular height information of the different atlas images of the sample structure atlas with respect to one another. The sample position information is information about the position of the sample structures, captured in the atlas images, in the sample of the sample type, in particular the position along a plane parallel to the focus position.

Due to the fact that the sample structure atlas comprises both sample position information and focus position information, the target sample structure, captured with the target image, in a sample of the respective sample type can be directly approached by the imaging device based on an atlas image selected as target image and the focus position of the imaging device can be suitably repositioned accordingly.

Preferably, the determining of the compare signature comprises recording a compare stack comprising a plurality of images with compare focus positions being height offset to one another. The determining of the signature distances respectively comprises determining a signature distance between the compare signatures based on the images of the compare stack and the target signature. The determined signature distances are, in particular, distances in the embedding space. In the sense of the present invention, signature distances are to be understood here as distances between signatures, or result outputs, determined by means of a suitable distance measure or by means of a similarity measure. Suitable distance and similarity measures comprise, for example: a Euclidean distance and a Manhattan distance. The compare stack comprises at least one image, the images of the compare stack are also called compare images.

Because the compare stack is recorded during the experiment when determining the compare signature and the signature distances between the compare signatures based on the images of the compare stack and the target signature are respectively determined when determining the signature distances, among the images of the compare stack the image with the optimal focus position can be determined based on the minimum signature distance between compare signature and target signature. The present invention thus provides a particularly simple possibility for finding or repositioning the focus position into the target focus position by recording the compare stack and correspondingly determining the signature distance.

Preferably, the recording of a compare stack takes place as soon as a signature distance between the target signature and a compare signature based on an image recorded with the current focus position is greater than a predetermined value. Unnecessary recordings of a compare stack can thereby be avoided, which reduces a light exposure of the samples without impairing the quality of the experiment, since the focus position of the images recorded in the course up to then still coincides well with the target focus position.

Preferably, the repositioning of the focus position takes place in a plurality of repositioning rounds, wherein in particular the height offset of the images of the compare stack to one another is reduced in successive repositioning rounds.

As the height offset of the images of the compare stack to one another is reduced in successive repositioning rounds, the focus position of the imaging device can be brought closer and closer to the target focus position.

Preferably, the adapting of the current focus position comprises determining a new focus position and adapting the current focus position to the new focus position. The determining of the new focus position takes place based on the signature distances between the compare signatures and the target signature, wherein in particular a focus position corresponding to a smallest signature distance between compare signature and target signature is selected as new focus position. If the signature distance is determined by means of a degree of correspondence, a focus position corresponding to a greatest correspondence between compare signature and target signature can alternatively be selected as the new focus position.

Preferably, the determining of the new focus position comprises in particular selecting the compare focus position corresponding to the compare signature with the smallest signature distance as new focus position and/or calculating the new focus position based on at least one determined signature distance and a correspondence between signature distance and height offset.

Due to the fact that the new focus position is determined based on the compare signature with the smallest signature distance to the target signature, the repositioning of the focus position can be implemented in a simple manner.

Preferably, the calculating of the new focus position comprises calculating a height offset based on a determined distance transformation, wherein the distance transformation maps a signature distance between two signatures output by the machine learning model to a corresponding height offset of the focus positions of the underlying images, wherein the distance transformation was in particular determined from a sample of the sample type.

Due to the fact that the new focus position is determined based on the distance transformation, a load on the sample during the holding or repositioning of the focus position of the imaging device can be further reduced, since the new focus position can already be well determined from fewer or only a single recorded compare image based on the signature distance between the compare signature and the target signature and the distance transformation.

Preferably, the machine learning model comprises one or more of the following:
a machine learning model initialized with random weights,
a machine learning model pre-trained with non-specific image data, wherein the machine learning model pre-trained with non-specific image data has in particular been trained for identifying objects with non-specific image data, and
a machine learning model pre-trained on specific image data, wherein the specific image data in particular comprise image data from a previous experiment with a sample of the same sample type or image data from a sample structure atlas.

Due to the fact that a machine learning model with randomly initialized weights can be used for outputting the signature, a training of the machine learning model is not necessary and the method can be applied directly.

Due to the fact that a machine learning model pre-trained with non-specific image data is used, the signatures of different sample structures of interest can be better separated from one another, since the pre-trained machine learning model has already been trained for identifying different structures.

Due to the fact that a machine learning model pre-trained with specific image data is used for outputting the signature, for example, the machine learning model can also be used for identifying sample structures of interest, wherein the machine learning model is implemented as a classification model, also called a classifier, and an output of an output layer of the machine learning model classifies the input images according to contained sample structures. An intermediate output of one or more intermediate layers of the machine learning model can in particular be feature maps which are used as signature. The present invention thus achieves the fact that a machine learning model for several objects is used in the method for repositioning an imaging device.

Preferably, a training of the machine learning model comprises one or more of the following:
an unsupervised learning,
a supervised learning,
training the machine learning model for classifying image data,
training an embedding model comprised by the machine learning model for embedding into an embedding space, wherein the embedding model is trained to map embeddings of images with height-offset focus positions into the embedding space such that a height offset of the images with height-offset focus positions corresponds to a signature distance of the embeddings of the images in the embedding space, and
training a metric model comprised by the machine learning model, wherein the metric model is trained to respectively assign a signature distance to two input signatures such that the height offset of the height-offset focus positions is mapped to a corresponding signature distance, wherein the metric model can for example be implemented as a regressor which directly outputs the signature distance, or the metric model embeddes the input signatures into a feature space in which the signature distance is determined from the embedded signatures by means of a distance measure.

Due to the fact that the machine learning model can be trained differently, the method is particularly flexible and can be adapted according to the requirements.

The unsupervised training comprises in particular a training of an autoencoder which comprises an encoder network and a decoder network. The encoder network outputs the signature here. During the inference, only the encoder network which outputs the signatures is used.

Preferably, the training of the machine learning model comprises a training of a main task and a training of an auxiliary task, wherein the main task comprises the outputting of the signature and the auxiliary task comprises one or more of the following auxiliary tasks:
classifying the image data, in particular classifying the image data on the basis of sample structures contained in the image data,
semantic segmenting of the image data,
classifying the image data, wherein the image data are divided at least into image data with sample structures of interest and image data without sample structures of interest,
carrying out a transformation,
a pseudo-task, in which a random noise is added to calculated gradients during the training.

By combining the training of a main task, here the training of the outputting of the signature, with an auxiliary task, the training can be improved in particular for training data with only a few annotations.

Preferably, the transformation comprises in particular one or more of:
a mirroring,
a rotation,
a scaling,
a deformation by means of an elastic grating,
a brightening,
a darkening,
a setting of the gamma correction value,
a vignetting,
an offset,
a color inversion,
an artificial noising,
a denoising,
a sub-sampling,
a masking,
a blurring,
an arbitrary filtering with a linear or non-linear filter,
a focusing,
an artefact distance mapping,
a deconvolution,
a down-sampling, and
an inpainting of the microscope image, wherein the transformation is in particular carried out by means of a trained processing model.

According to some embodiments of the present invention, a detector, also called detection model, is a machine learning model which has been trained to identify predetermined detection patterns in input data and to output a list on the basis of which the identified detection patterns can be read out or determined and further processed. In particular, the list is a list of localizations, for example a localization in the input data. The input data can be in particular an image, an image stack or else an input tensor. The precise format of the localization depends in particular on the input data and the detection patterns to be identified, in particular on the format of the input data and the detection pattern to be identified.

According to some embodiments of the present invention, classifiers, also called classification models, have been trained or are set up to assign a class to an input datum. The output datum can be in particular a class assigned to the input datum, wherein the format can be in particular a vector, wherein each entry of the vector corresponds precisely to one of the possible classes to be assigned and in particular a “1” entry in the vector indicates the class. Alternatively, a class number can also be output, for example. Alternatively, however, a classifier can also be trained such that a vector is output, wherein the entries in the vector in each case indicate a probability that the respective input datum belongs to the class corresponding to the entry of the vector. Depending on an implementation, the respective format of the output datum varies from classification models and correspondingly also the format of the target datum varies in an annotated data record for training the classification model.

A semantic segmenter, also called semantic segmentation model, in the sense of some of the embodiments of the present invention is an image-to-image image which assigns an output value corresponding to a semantic to each pixel or each image region of an input datum, i.e. the output datum is also once again an image, the output image is also called semantic segmentation mask or only segmentation mask.

Preferably, the machine learning model is in particular a multi-layer perceptron, MLP, a recurrent network, a transformer network, in particular a convolutional neural network, an autoencoder, an encoder, a decoder, an encoder-decoder model, a transformer, a classification network, in particular a ResNet, DenseNet or EfficientNet or an active appearance model.

Preferably, the method also comprises selecting the machine learning model from a series of pre-trained machine learning models, in particular automatically selecting the machine learning model based on a first image recorded by the sample, an overview image and/or context information.

Preferably, the context information comprises
a type of the sample which is imaged in the images,
an experiment carried out,
an imaging device used,
a type of the sample carrier which was used, for example whether a chamber slide, a microtiter plate, a slide with cover glass or a Petri dish was used,
image recording parameters, such as, for example, information about illumination intensity, exposure duration, filter settings, fluorescence excitation, contrast method or sample table settings,
information about objects contained in the respective image,
application information which indicates for which type of application the images were recorded,
information about a user who has recorded the images.

A further aspect consists in a method for training a machine learning model for outputting a signature, wherein the signature is suitable for being used in the above-described method for repositioning a focus position. The training of the machine learning model thus permits a focus position in experiments even with thick samples to be suitably adjusted.

A further aspect relates to an evaluation device for repositioning a focus position of an imaging device, comprising means for carrying out the above-described methods, and to an evaluation device for carrying out the above-described method for training a machine learning model.

A further aspect of the invention relates to a repositioning system comprising the evaluation device.

A further aspect relates to a computer program product, comprising commands which, when the program is executed by a computer, cause the latter to carry out one of the above-described methods, the computer program product being, in particular, a computer-readable storage medium.

### BRIEF DESCRIPTION OF THE FIGURES

The invention is explained in more detail below on the basis of the examples illustrated in the drawings. The drawings show in:

FIG. 1 schematically a system for use with the method for repositioning a focus position of an imaging device into a target focus position according to one embodiment;

FIG. 2 schematically a device for use with the method according to one embodiment;

FIG. 3 schematically a method for repositioning a focus position of an imaging device into a target focus position according to one embodiment;

FIG. 4 schematically a machine learning model for use with the method according to one embodiment;

FIG. 5 schematically a conventional method for repositioning a focus position according to the prior art;

FIG. 6 schematically a further conventional method for repositioning a focus position according to the prior art;

FIG. 7 schematically a method for repositioning the focus position of an imaging device into a target focus position according to one embodiment.

### DETAILED DESCRIPTION OF THE EMBODIMENTS

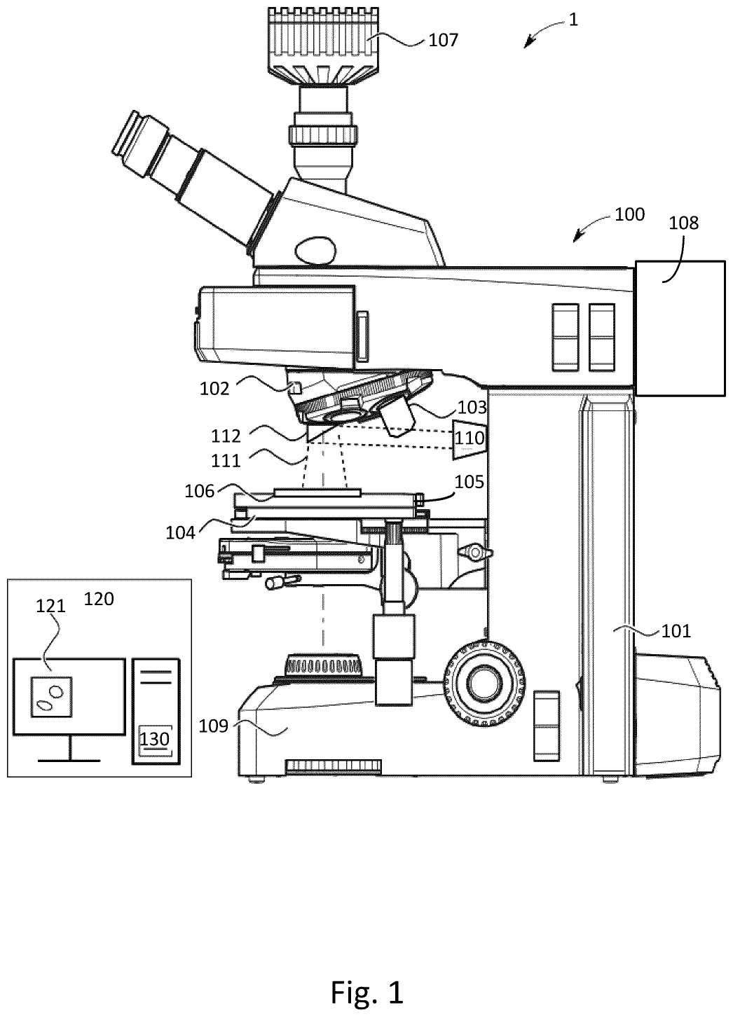

An exemplary embodiment of a repositioning system 1 comprises a microscope 100, also called imaging device, a control device 120 and an evaluation and control device 130, in short evaluation device 130. The microscope 100 is communicatively coupled to the evaluation device 130 (for example to a wired or wireless communication link). The evaluation device 130 can evaluate image data 220 captured with the microscope 100 (FIG. 1) and control the microscope 100, for example, on the basis of the evaluated image data 220. If the repositioning system 1 comprises a machine learning model 140, for example a neural network, it is also referred to as a machine learning system.

The microscope 100 according to the illustrated embodiment is a light microscope. The microscope 100 comprises a stand 101 which comprises further microscope components. The further microscope components are in particular an objective changer or objective turret 102 with a mounted objective 103, a sample stage 104 with a holding frame 105 for holding a sample carrier 106 and a microscope camera 107.

If a sample is clamped into the sample carrier 106 and the objective 103 is pivoted into the microscope optical path, a fluorescence illumination device 108 can illuminate the sample for fluorescence recordings, the microscope camera 107 can receive the fluorescence light as detection light from the clamped sample and record image data 220 in a fluorescence contrast. If the microscope 100 is to be used for transmitted light microscopy, a transmitted light illumination device 109 can be used in order to illuminate the sample. The microscope camera 107 receives the detection light after passing through the clamped sample and records image data 220. Samples can be any desired objects, fluids or structures.

The sample carrier 106 and the objective 103 are mounted so as to be movable relative to one another along an axis, the axis along which the relative movement takes place is usually equated with the z axis or the z direction, and a change in the relative position with respect to one another is also referred to as the z drive. The sample stage 104 is usually arranged so as to be movable along the z axis, but there are also microscopes in which the sample stage 104 is fixed and the objective 103 is arranged so as to be movable along the z axis.

The microscope 100 optionally comprises an overview camera 110 with which overview images of a sample environment can be recorded. The overview images show the sample carrier 106, for example. A field of view 111 of the overview camera 110 is larger than a field of view during a recording of image data 220 with the microscope camera 107. The overview camera 110 looks at the sample carrier 106 by means of a mirror 112. The mirror 112 is arranged on the objective turret 102 and can be selected instead of the objective 103.

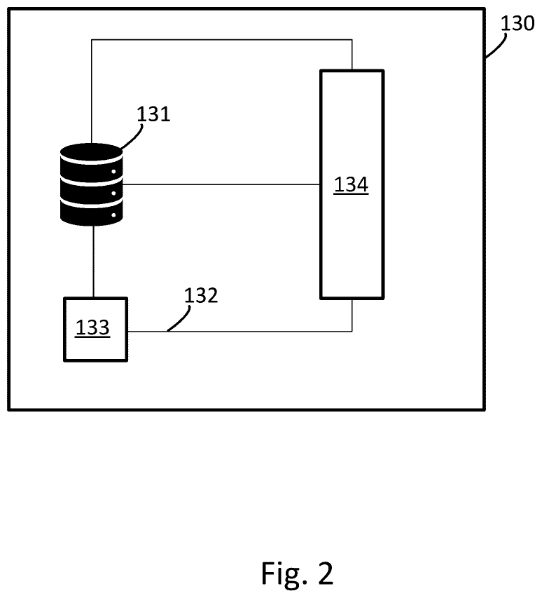

According to this embodiment, the control device 120, as illustrated schematically in FIG. 1, comprises a screen 121 and the evaluation device 130. The control device 120 is configured to control the microscope 100 to record image data 220 and to store the image data 220 recorded by the microscope camera 107 on a memory module 131 (see FIG. 2) of the evaluation device 130. The recorded image data 220 can be displayed on the screen 121 if required. The evaluation device 130 is configured to process the recorded image data 220.

The evaluation device 130 comprises various modules which exchange data via channels 132. The channels 132 are logical data connections between the individual modules. The modules can be designed both as software modules and as hardware modules.

The evaluation device 130 comprises the memory module 131. The memory module 131 stores the image data 220 recorded by the microscope 100 and manages the data to be evaluated in the evaluation device 130.

The evaluation device 130 comprises the memory module 131, by means of which image data 220 are stored and stored. A control module 133 reads image data 220, that is to say image stacks comprising microscope images or a plurality of microscope images being height offset to one another, for example compare stacks, and a target signature or atlas images from the memory module 131 and forwards the read-out data to an analysis module 134. According to one embodiment, the control module 133 reads the image data 220 and inputs them into the analysis module 134.

According to some embodiments of the present disclosure, the repositioning system 1 is designed to reposition a focus position of the imaging device 100 based on a target focus position.

The analysis module 134 evaluates the input image data 220 and forwards control information to the control module 133 on the basis of the evaluation or forwards the results of the evaluation to the memory module 131 for storage. Based on the control information, the control module 133 controls the microscope 100 to reposition the focus position of the microscope 100, such that the microscope 100 as good as possible holds the target focus position or corrects it such that the focus position of the microscope 100 is as close as possible to the target focus position.

The control information can in particular be information about a position of the target focus position, on the basis of which the control module 133 controls the focus position of the microscope 100 into the target focus position, or it can be directly information for controlling the microscope 100, which is forwarded by the control module 133 to the microscope 100.

In particular, the analysis module 134 can comprise a machine learning model 140. In particular, the machine learning model 140 is implemented as a neural network. During the training of the machine learning model 140, the control module 133 controls the analysis module 134 such that the analysis module 134 reads some of the data of an annotated data set from the memory module 131 and inputs it into the machine learning model 140. The analysis module 134 determines an objective function on the basis of the output data of the machine learning model 140 and based on target data of the annotated data set and optimizes the objective function by adapting the model parameters of the machine learning model 140 based on the objective function.

In particular, the optimization of the objective function is carried out by means of a stochastic gradient descent method. In the stochastic gradient descent method, only a small subset of the training data of the annotated data set, referred to as batch, is used in each case. The control module 133 determines an objective function for each input datum of the batch on the basis of output datum 240 output by the machine learning model 140 and the target datum, corresponding to the input datum 220, of the annotated data set, which objective function captures a difference between the output datum 240 and the target datum. The control module 133 then calculates a gradient for each of the calculated objective functions with respect to the model parameters of the machine learning model 140, sums the calculated gradients over the batch and determines the mean value. The control module 133 determines updated model parameters for the machine learning model 140 from the mean value by so-called back propagation. The machine learning model 140 is newly initiated by the control module 133 with the updated model parameters in the analysis module 134, and a next step of the stochastic gradient descent method is carried out.

The training of the machine learning model 140 ends as soon as it is achieved by the optimization of the objective function that the objective function reaches a predetermined limit value.

If the training has been completed, the control module 133 stores the last used model parameters of the machine learning model 140 in the memory module 131, in particular together with context information, so that the machine learning model 140 just trained can be identified again later and can be initialized, for example, for further training or the inference.

As an alternative to the stochastic gradient descent method, other methods can also be used. In particular, any other training method can be used.

According to some embodiments of the present invention, the control module 133 can either initialize a randomly initialized machine learning model 140, initialize a machine learning model 140 trained on the basis of non-specific data or initialize a machine learning model 140 completely trained on the basis of specific image data in the inference. If the machine learning model 140 is initialized in the analysis module 134, the control module 133 reads the image data from the memory module 131 during the processing and forwards them to the analysis module 134.

The analysis module 134 processes the input data, wherein the analysis module 134 inputs in particular image data 220 as input data into the machine learning model 140, in particular into an input layer 141, and further processes the output data 240 of the machine learning model 140. In particular, the outputs of the machine learning model 140 comprise the output data 240 of the output layer 143 and in particular intermediate outputs 230 of intermediate layers 142 of the machine learning model 140. The intermediate outputs 230 can in particular be used as signature 250.

The analysis module 134 analyzes the output data 240, in particular the output signatures 250, and determines a new focus position based on a signature distance between output compare signatures and a target signature. Information about the new focus position is forwarded to the control module 133 as control information. Based on the control information, the control module 133 repositions the focus position of the microscope 100.

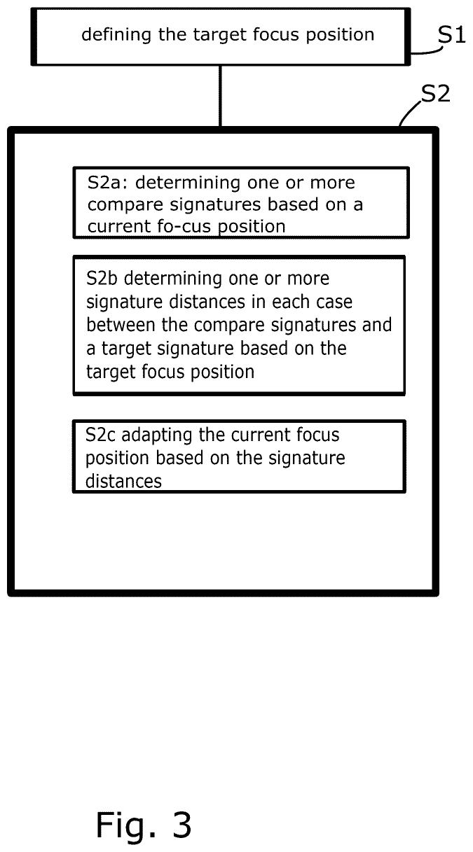

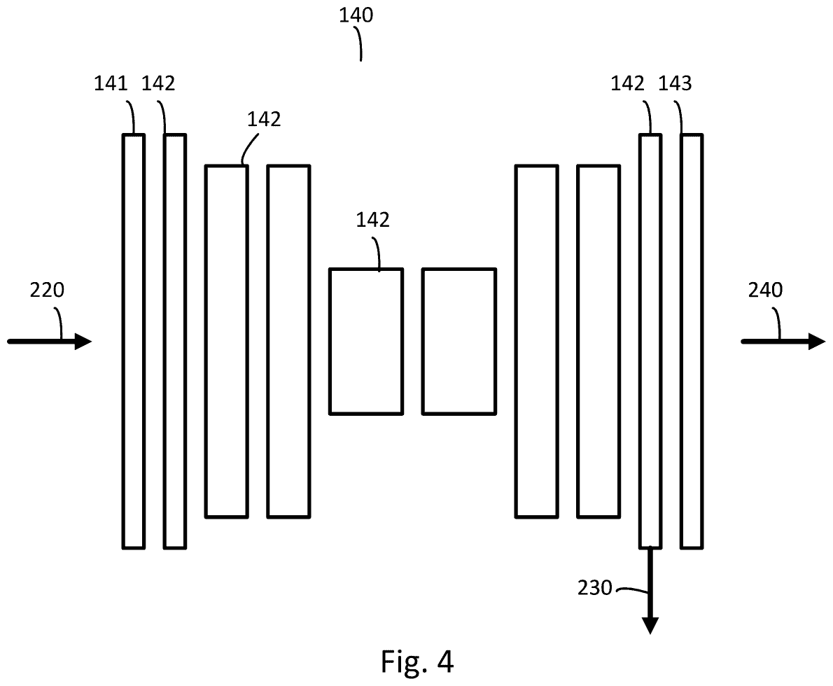

A method for repositioning a focus position of an imaging device, in this case the microscope 100, based on a target focus position is described below with reference to FIGS. 3 to 7.

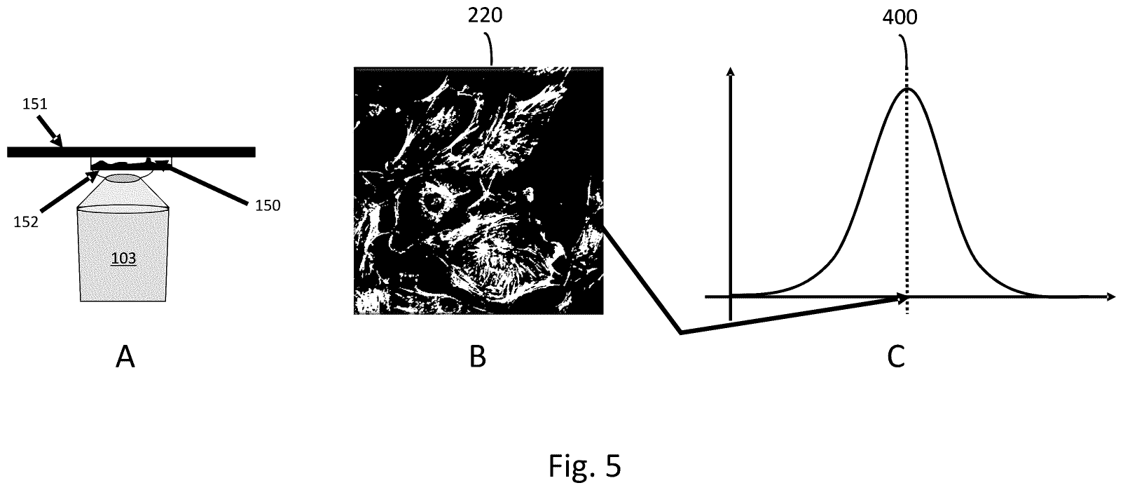

Software-based holding focus for microscopes 100 is known from the prior art, see FIG. 5. In the case of the software-based holding focus, the target focus position is the focus position with the greatest image sharpness. In the prior art, the software-based holding focus is applied to thin samples 150. Thin samples 150 can be inserted, for example, between a slide 151 and a cover glass 152 in holding frames 105. In the thin sample 150, an image sharpness along a height of the sample is determined by an evaluation device 130. In an image sharpness profile 400 illustrated in partial figure (C), in which the image sharpness is plotted against the position along the height, a global maximum results for thin samples. The maximum, that is to say the focus position with the maximum image sharpness, corresponds precisely to the target focus position in experiments with thin samples. In the course of an experiment, the focus position of the microscope 100 is repeatedly corrected or repositioned by an evaluation device 130 based on an image sharpness of a current focus position to the target focus position; this can be carried out, in particular, by changing the focus position and then continuing to move in the direction of the increasing image sharpness until the focus position with the maximum image sharpness is reached again, in particular at the global maximum.

In the described method for repositioning a focus position, the target focus position in the sample is first defined for an experiment in a step S1.

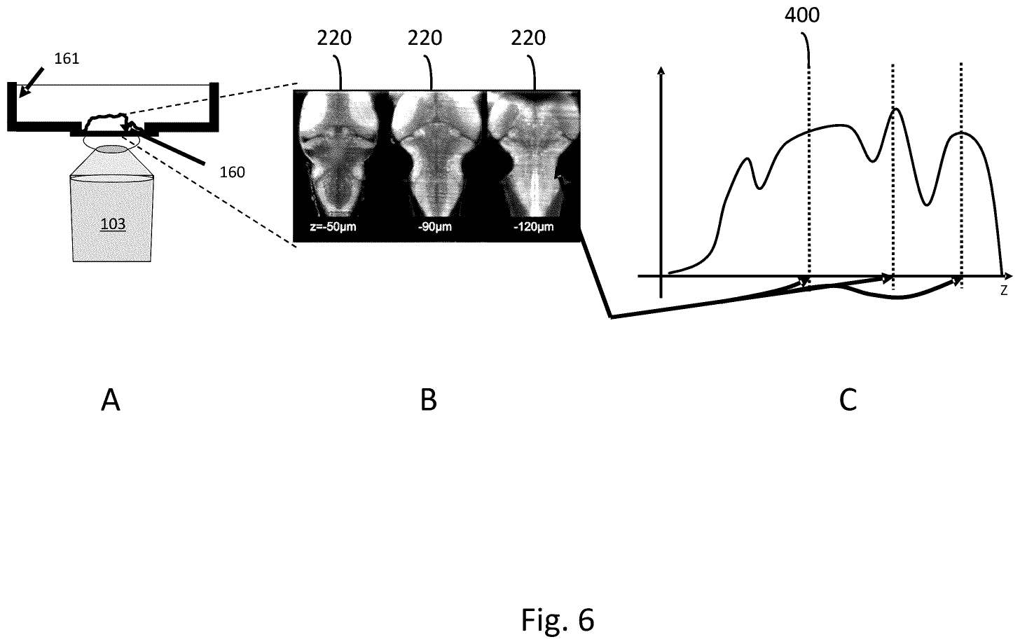

In contrast to the prior art described above, the method for repositioning a focus position according to some embodiments of the present invention is used for samples, in particular thick samples 160, as illustrated schematically in FIG. 6A, in which there are sample structures 300 to be examined which are of interest at different focus positions. Thick samples 160 can be stored, for example, in Petri dishes 161 and examined in the microscope 100. FIG. 6B shows, for example, three sections at different heights of a sample, z=−50 μm, z=−90 μm and z=−120 μm, which each capture sample structures 300 of interest (in focus). Furthermore, FIG. 6C shows the image sharpness profile 400 of the thick sample along the height of the sample, that is to say for the different focus positions of the microscope 100 offset in height along the z direction. The dashed lines in FIG. 6C correspond precisely to the positions of the sections illustrated in FIG. 6B. If the focus position of the microscope 100 is set precisely to these positions, the illustrated sections are imaged in focus by the microscope 100. If one of these sections corresponds precisely to the target sample structure 300, the position of the dashed line would correspond precisely to the target focus position. As can be seen in the image sharpness profile 400 in FIG. 6C, the different focus positions of the different sections correspond only partially to local maxima. The sections at z=−90 μm and z=−120 μm correspond precisely to local maxima here, but the section at z=−50 μm corresponds precisely to no local maximum. In the case of common software-based holding focus methods, the focus position is held on the basis of a global maximum; therefore, it would not be possible for the sections illustrated in FIG. 6B to hold or reposition the focus position on the respective focus positions of the sections in a software-based manner.

According to a first embodiment, the defining of the target focus position is carried out by recording a target image of a sample to be examined. The target signature is determined by inputting the target image into the machine learning model 140. In particular, a target sample structure 300 is imaged in focus by the microscope 100 in the target image.

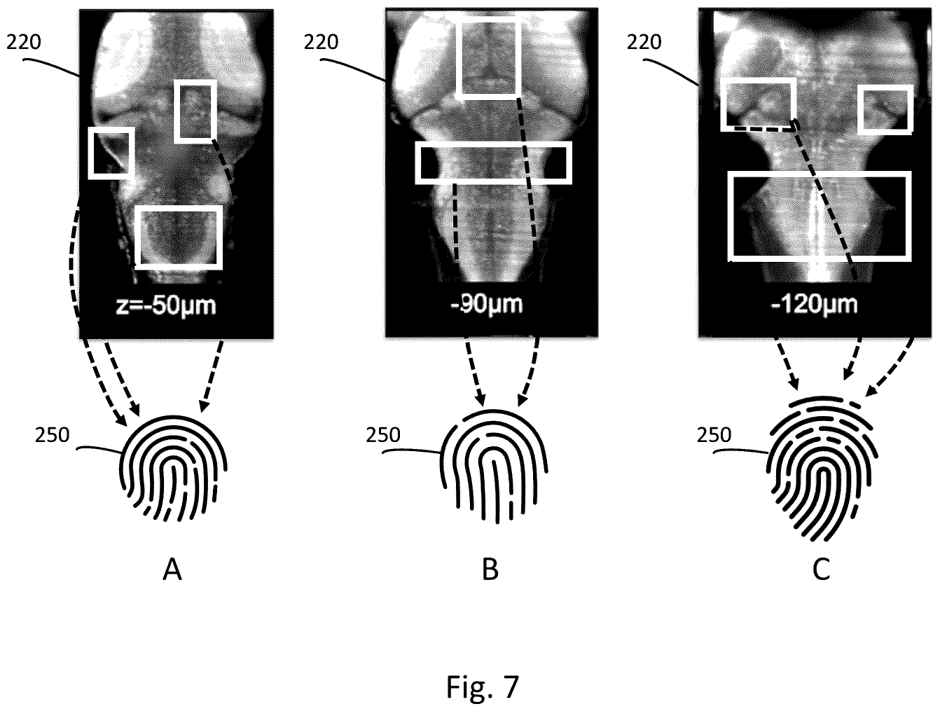

FIG. 7 show in each case schematically different signatures 250 in the partial figures (A), (B) and (C) for the sections described above with reference to FIGS. 5 and 6. According to the illustration in FIG. 7, the signatures 250 are illustrated here as fingerprints only for better understanding. Likewise for better understanding, the sample structures 300 to be examined in the different sections are each bordered with boxes in FIG. 7. This is intended only to illustrate which regions of the illustrated sections are illustrated in focus here. Since the signatures 250 output by the machine learning model 140 are in particular feature maps of intermediate layers 142 of the machine learning model 140, these feature maps can be selected precisely such that they react particularly sensitively to specific structures in the imaged regions of an image. The structures can be imaged in particular particularly sharply in comparison to the surrounding structures.

According to the first embodiment, the machine learning model 140 is a randomly initialized neural network, in particular a classifier, for example a ResNet. The target signature is an intermediate output 230 of an intermediate layer 142, in particular an embedding layer. Alternatively, the target signature can also be composed of a plurality of intermediate outputs 230 of a plurality of intermediate layers 142. After the machine learning model 140 has output the target signature, the target signature can be stored in the memory module 131 for the further course of the experiment.

According to the first embodiment, step S1 is followed by step S2, repositioning a current focus position of the imaging device, based on the target focus position.

Step S2 is composed of a plurality of substeps which are carried out repeatedly.

In a step S2a, firstly one or more compare signatures are determined. For determining the compare signatures, the microscope 100, starting from the current focus position, records a plurality of microscope images having height-offset focus positions to one another. For this purpose, the control module 133 controls the microscope 100 such that the microscope 100, starting from the current focus position, records an image in the current focus position, and, height-offset above the current focus position and height-offset below the current focus position, records one or more images, for example 1, 2, 3 or 4, or 10, or up to 20 images, the compare stack. For each of the images of the compare stack, the evaluation device 130 determines a compare signature by means of the machine learning model 140.

In a step S2b, the analysis module 134 determines a signature distance from the target signature for each of the compare signatures. The compare signatures output by the machine learning model 140, like the target signature, form an intermediate output 230 of an intermediate layer 142 of the machine learning model 140. These intermediate outputs 230 can be interpreted as embeddings into an embedding space. By means of a suitable distance measure, the signature distance of the different compare signatures to the target signature can be determined in this embedding space. Based on the signature distances, the analysis module 134, after the determining of the signature distances, determines the image whose compare signature has the smallest signature distance and determines a new focus position based on the smallest signature distance, wherein the new focus position corresponds precisely to the focus position of the image of the images in the compare stack whose compare signature has a smallest signature distance to the target signature 250.

By way of example, a Euclidean distance in the embedding space, also referred to as L2 standard, can be used as distance measure. Alternatively, a Manhattan distance (L1 standard) can also be used or any other desired L standard. Any desired distance measures can be used here. Alternatively, a signature distance can also be determined by means of a similarity measure or a degree of correspondence. In particular, a kernel function or a correlation coefficient comes into consideration as a similarity measure.

In a last substep S2c of step S2, the focus position of the microscope is set to the new focus position. The signature 250 of the image corresponding to the new focus position has the smallest signature distance to the target signature, for which reason the new focus position corresponds best to the target focus position.

According to one configuration, step S2 for repositioning the focus position is carried out again after step S2 has been carried out. In particular, with each newly executed step S2, during the renewed recording of a compare stack, a height offset between the height-offset images of the compare stack is reduced in comparison with a previous step S2. Based on the newly recorded compare stack, the substeps S2b and S2c are then carried out again, with the result that the signature distance between one of the newly determined compare signatures and the target signature is lower with a high probability than in a previous step S2, for which reason this new focus position is with a high probability closer to the target focus position than in a previous step S2.

According to one configuration, for example, with a predetermined time interval, in each case the substeps of step S2 are carried out repeatedly, as described above, for example in each case with a decreasing height offset between the height-offset images of the compare stack.

According to a further configuration, in substep S2a, initially only one image can also be recorded in the present focus position of the microscope 100. Then, based on the image recorded in the present focus position, a compare signature of the image is determined and only if the signature distance between the compare signature and the target signature is greater than a minimum amount, substep S2a for recording the compare stack is carried out, i.e. after the recording of the image in the present focus position of the microscope 100, further images are recorded which lie below and above the present focus position of the microscope 100.

According to a further configuration, in step S2c, the new focus position can also be determined based on the determined distances and the different compare focus positions to a focus position between the different compare focus positions.

According to further configurations of the first embodiment, the machine learning model 140 can be in particular a multi-layer perceptron, MLP, a recurrent network, a transformer network, in particular a convolutional neural network, an autoencoder, a classification model, also called classification network, in particular a DenseNet or EfficientNet or an active appearance model.

In particular, the machine learning model 140 can be implemented as a classification model, segmentation model or detection model which is trained, for example, on the basis of non-specific training data or on the basis of specific training data for identifying specific structures. For example, the image data of ImageNet can be used as non-specific training data. Alternatively, image data from previous experiments, or also other sources, can be used as training data; the machine learning model 140 is then trained in particular for identifying sample structures 300, i.e. specific structures in the respective sample. In particular, the machine learning model 140 can be trained on the basis of a sample structure atlas, then the image data of the sample structure atlas form the training data.

A second embodiment of the repositioning system 1 likewise comprises the microscope 100, the control device 120 and the evaluation device 130. A method carried out by the second embodiment of the repositioning system 1 comprises a step S1 of defining the target focus position and a step S2 for repositioning the target focus position. According to the second embodiment, the machine learning model 140 comprises a metric model. The metric model according to the second embodiment differs from the machine learning model 140 in that the outputting of the signatures is trained such that the signatures, that is to say the outputs of the metric model, are embedded into an embedding space such that a distance of the signatures, referred to as a signature distance, can be determined in the embedding space. A space with a distance measure is also referred to as metric space or space with a metric.

The training is carried out, for example, with image data recorded in a previous experiment or with image data of a sample structure atlas; in particular, the machine learning model 140 can be trained in a sample-type-specific manner; alternatively, however, a machine learning model 140 can also be trained for all possible different sample types and experiments. In particular, the embedding is configured such that the signature distance corresponds to the height offset of the focus positions of the underlying images, based on the correspondence between a height offset of the focus positions of two images and the signature distance, the height offset of the images to one another can be determined from the signature distance of two signatures and, correspondingly, the height offset between the compare focus position with which the respective compare image was recorded and the target focus position can be determined from a distance of a compare signature to the target signature and thus correct the focus position according to the height offset in order to readjust the microscope 100 to the target focus position along the z-axis. For example, a distance transformation between the height offset and the signature distance can be determined, by means of which a height offset can be determined from a determined signature distance. If the correspondence between height offset and signature distance is linear, then a signature distance can be converted into a height offset by means of a proportionality factor to be determined.

During the training of an embedding model, a so-called triplet-loss can be used as an objective function, for example. The triplet-loss is configured such that it captures a distance between two embeddings, wherein a positive example and a negative example are embedded and, in the training, the model parameters are adapted based on the objective function such that the positive example is mapped onto an anchor embedding, while the negative example is embedded into the embedding space at a predetermined signature distance from the anchor embedding.

Applied to this embodiment, the positive example would be, for example, an image of the target sample structure recorded in the target focus position, the embedding anchor would be the target signature and an image of the sample offset in height by a certain height offset “d” in relation to the target focus position would be the negative example. In the training, for example, by means of a stochastic gradient descent method and back propagation, the model parameters of the embedding model would be adapted such that the image of the target sample structure recorded in the target focus position is mapped onto the target signature into the embedding space with as small a distance as possible, while the embedding of the image offset in height in relation to the image recorded in the target focus position with the height offset “d” is precisely mapped into the embedding space such that it is mapped onto a signature which has the predetermined signature distance “D” in relation to the target signature in the embedding space, wherein a suitable correlation exists between the height offset “d” and the signature distance “D”. For example, the embedding can be carried out such that the height offset “d” and the signature distance “D” have the same numerical value, then a proportionality factor which reproduces the correspondence is precisely one. However, any other proportionality factor or any other desired distance transformation between the height offset and the signature distance is also possible.

Furthermore, image stacks comprising the target image with mutually registered and respectively height-offset focus positions can be used in the training. If the images of the image stack each have, for example, a height offset of “d” and the target focus position lies in the middle of the image stack, then in the training, the images recorded above and below the target focus position can each be embedded relative to the target signature with a distance “D” into the embedding space, but in addition the embedding of the further images of the image stack can also be trained to one another such that images of the image stack which likewise have the height offset of “d” to one another have a signature distance of “D” in the embedding space, images whose focus positions have, for example, a height offset of “2d”, that is to say twice the height offset of neighboring images of the image stack, have a signature distance of “2D” in the embedding space, that is to say twice the signature distance compared with the signature distance of neighboring images of the image stack. The same applies correspondingly to further images of the image stack with, if appropriate, even greater height offsets.

If the training of the embedding model is ended, after the recording of a compare image in step S2b of the second embodiment, the signature distance in the embedding space can be determined, for example, by means of the distance measure from the compare signature and the target signature. Since the signature distance corresponds to a determined height offset, the height offset of the compare focus position of the compare image to the target focus position can be determined or calculated based on the signature distance and the distance transformation or based on the proportionality factor, and the new focus position can be determined or calculated from the height offset.

The described training of the embedding model thus makes it possible to find or hold the target focus position very precisely with few recordings of compare images. In particular, the images recorded by the sample in a continuous method of the focus position are imaged onto a continuous path through the embedding space. This type of optimization of the embedding is also referred to as convex minimization. The height offset with respect to the target signature can in turn be determined from a position in the embedding space respectively, and the z drive, that is to say the focus position, of the microscope can be adapted correspondingly to the target focus position.

According to one configuration of the second embodiment, step S2 comprises inputting a compare signature based on a current focus position and the target signature into a metric model, wherein the metric model maps or embeddes the input signatures 250 into a feature space such that a signature distance of the embedded signature corresponds to the height offset between the current focus position and the target focus position. Based on the signature distance, the new focus position can then be correspondingly determined again and the microscope can be set to the new focus position.

In particular, the metric model is trained on the basis of previous experiments of samples of the same sample type. For this purpose, for example, at the start of an experiment with a sample of a sample type, the sample is scanned along the height and images of the sample are recorded in each case in different focus positions of the microscope 100. From the images, signatures 250 are respectively determined by the machine learning model 140 and, based on the signatures 250 corresponding to the respective focus positions, a metric model is trained such that it maps the signature distance of the signatures 250 embedded into the feature space to the height offset of the different corresponding focus positions along the height or the distance transformation can be determined, on the basis of which the height offset of the current focus position to the target focus position or the height offset of two images with different focus positions with respect to one another can be determined from the signature distance of the embedded signatures 250 in the feature space.

In the training of metric models, the parameters of the trained metric model are adapted such that a mapping of the input signatures 250 into a feature space takes place such that a distance of two signatures 250 embedded into the feature space in the feature space corresponds to a signature distance of the focus positions corresponding to the two signatures 250 and thus a proportionality factor can be determined such that the height offset of the focus positions of the respective images or signatures can be determined from a determined signature distance and the proportionality factor.

According to a further configuration, an actual signature can be determined based on a single image recorded in the present focus position of the microscope 100 and the new focus position can be determined based on a signature distance of the actual signature to the target signature. Here, a height offset in the z direction is calculated based on the signature distance and the distance transformation, and the new focus position is determined based on the height offset.

According to a further configuration of the second embodiment, the first and the second embodiment can be combined. For this purpose, as in the first embodiment, the focus position of the microscope 100 in the sample is first repositioned to the target focus position by means of steps S2a, S2b and S2c of the first embodiment. Based on the compare stacks recorded in each case in substep S2a, the metric model according to the second embodiment is then trained as described. It can be assumed that the correspondence between the height offset and the signature distance becomes better with each additionally recorded compare stack and thus the accuracy of the distance transformation determined therefrom is also improved.

According to a further configuration of the first and the second embodiment, for example, at the start of the experiment with a sample of a sample type or if a current focus position deviates greatly from the target focus position, the height offset of the images in the compare stack can initially be selected to be relatively large, as a result of which a large region of the sample is detected or verified, applied to the first embodiment, it is thereby ensured that one of the images of the compare stack has a compare signature which lies in the vicinity of the target signature. In the further course of the experiment, in particular with approaching the target focus position, the height offset of the images in the compare stack can be reduced. Applied to the first embodiment, by means of the reduction of the height offset, the sample is sampled more and more finely, as a result of which it is possible to approach the target focus position ever better. Applied to the second embodiment, in particular to the combination of the first and the second embodiments, by means of the reduction of the height offset, the embedding in the vicinity of the target signature can be trained with a higher precision, for which reason the distance transformation between the height offset and the signature distance in the vicinity of the target signature images particularly exactly between the spaces and the target focus position can thus be set particularly exactly.

According to one configuration of the first two embodiments, the machine learning model 140 is for example a transformer network. In a transformer network, the individual images are respectively divided into image sections of equal size and linearized by means of a projection. The resulting, linearized projections, also called tokens, are written into a sequence. In addition, a position specification is supplemented for each of the linear projections, such that the sequence of the linearized projections comprises additional position information of the individual projections with respect to one another in each case. The projections are also preceded by an auxiliary projection, also called classification token, at the start of the sequence. The auxiliary projection can comprise, for example, loud zeros, an image generated by the encoder of the transformer can be used, for example, as a signature. Alternatively, an embedding of one or more of the projections, including the auxiliary projection, can also be used as signature.

According to a further configuration, the transformer network is in particular a visual transformer (e.g. vision transformer), transformer for short, which is initially pre-trained as a masked autoencoder, also called pre-training. For this purpose, individual tokens, that is to say individual ones of the linearized projections of the sequence, are masked randomly and the sequence including the masked tokens are input into the autoencoder. The outputs of an encoder of the autoencoder are input into a decoder of the autoencoder. The encoder comprises the transformer. The pre-training or the target function in the pre-training is configured such that the decoder attempts to reconstruct the masked tokens, that is to say the decoder is trained in the pre-training to reconstruct the masked linearized projections and thus ultimately the masked image sections again. If the pre-training is ended, the pre-trained transformer contained in the encoder can be trained as described above, for example for outputting the signature, in particular as described above with a triplet-loss, such that, after the training, the distance transformation between the height offset of focus positions of two images and a signature distance of the corresponding signatures in the embedding space can be determined. It has been shown that pre-training as a masked autoencoder positively influences the training of a transformer.

If the machine learning model 140 is an encoder-decoder network or an autoencoder, so-called bottleneck layers of the respective encoder; can be used to output the signature. Bottleneck layers are particularly suitable for outputting signatures, since they reduce the dimensionality of the input data and, as a result of this reduction, promote an extraction of particularly interesting characteristics of the data.

According to a further configuration, the training of the machine learning model 140 comprises a training of a main task and a training of an auxiliary task. The training of the main task can in particular comprise the outputting of the signature. The training of the auxiliary task can comprise any desired image processing not further related to the signature, for example a transformation, in particular any desired image transformations. Alternatively, the auxiliary task can also comprise a classifying or semantic segmenting, for example the image data can be classified or semantic segmented on the basis of the sample structures captured in the image data. In particular, the images can be classified or segmented on the basis of the biological structures contained in the images.

According to a further configuration, it can also occur that the embedding, that is to say the signatures, cannot be embedded in the embedding space such that there is a correspondence between the height offset and the signature distance in the embedding space. For this case, a distance agent can be trained by means of a reinforcement learning approach to suitably control an adaptation of the focus position.

During reinforcement learning, an agent is trained by interaction with the environment. The agent receives inputs about the state of its environment and decides, based on the current state and previous decisions, how the environment is influenced in a next step. Based on the result of the next step carried out, the agent receives a reward, a level of the reward depending on the quality of the step carried out.

Applied to the second embodiment, the distance agent, in this case a neural network, determines here how the focus position is to be moved along the z direction for recording compare stacks, in particular a number of height-offset images in the compare stack and their height offset to one another. After the recording of the compare stack, the distance agent receives for example the signature distances of the compare signatures to the target signature. The object for the distance agent can be for example to record as few compare stacks or compare stacks as possible with as few images as possible in order to arrive at the target focus position. Correspondingly, the reward is selected such that it is higher the fewer images the distance agent records in order to arrive at the target focus position. During reinforcement learning with neural networks, also referred to as deep Q learning, the model parameters are updated after each step or in each case after a plurality of steps by means of an objective function which in each case sensibly captures the reward, in order thereupon to decide better.

A third embodiment of the repositioning system 1 likewise comprises the microscope 100, the control device 120 and the evaluation device 130.

According to the third embodiment, no target image is recorded in step S1 for defining the target focus position, instead the target focus position is defined based on a sample structure atlas. Sample structure atlases are used in particular in biology. In a sample structure atlas, the different sample structures 300 in a sample are captured. In addition to the different sample structures 300, the sample structure atlas also comprises the focus position, corresponding to the respective sample structures 300, in the respective sample, referred to as focus position information, and in the case of extended samples can additionally also comprise a position along a plane perpendicular to the z axis, referred to as position information. In particular, a separate sample structure atlas must be provided in each case for samples of different sample types, for example different organisms, different cells or the like.

If the sample is a temporally variable sample, the sample structures 300 are also captured in the sample in terms of their change over time. For example, a specific sample can grow over an experiment, whereby a position of a sample structure 300 within the sample changes during the experiment, in particular also the focus position of the respective sample structure 300.

Furthermore, the target signature can also change during the experiment. In the first two embodiments, a new target image would therefore have to be recorded at regular intervals during the experiment, on the basis of which a new target signature would be determined. This is not necessary for samples for which a sample structure atlas is available, since the sample structure atlas captures the change over time of the different sample structures 300 in the sample with respect to the target signature and to the target focus position in the sample, in which the sample structure atlas accordingly comprises images for all regions of interest of a sample over the course or the development of the sample during an experiment.

According to one configuration, an embedding can also be carried out such that a signature distance has a plurality of components, in particular one component for each spatial direction. If the position of the respective structures of interest in all three spatial directions within the sample is also captured in the sample structure atlas, the sample can be correspondingly moved based on the different components of the signature distance such that the target focus position is correspondingly reached at the correct location in the sample. For this case, in addition to the focus position information which indicates a position of the respective sample structure along the z axis, the sample structure atlas also comprises sample position information which indicates the position of the respective sample structure along the further two spatial directions in the plane perpendicular to the z axis.

In the case of structures which are variable over time, it can also occur that a target sample structure develops further in the course of an experiment such that it has a plurality of components with different focus positions over the course. Correspondingly, the respective component of interest in the further course then has to be selected over the course and the target signature correspondingly defined, selected or determined.

In particular, a sample structure atlas can be created during an experiment, on the basis of which a focus position of a microscope 100 can be repositioned for later experiments in accordance with a selected target sample structure.

In particular, the third embodiment can also be combined with the first two embodiments; in particular, the repositioning of the target focus position based on the first embodiment can be carried out without a learned metric; alternatively, however, a metric can also be learned based on a sample structure atlas.

Sample structure atlases exist for a wide variety of fields of application. For example, in genetics and development biology, samples of the zebrafish embryo are frequently examined, since they have good optical properties. There are therefore extensive atlases for the zebrafish. There are also diverse atlases for further biological and a wide variety of clinical applications, for example in histology, these are often available online or also in printed form. Based on experiments carried out, a user can also himself create an atlas of his samples and then use it in later experiments.

For all three of the embodiments described above, it applies that the image data 220 processed by the machine learning model 140 can be in particular two-dimensional, for short 2D, that is to say images or image sections, three-dimensional, for short 3D, that is to say image stacks, but also four-dimensional, for short 4D, that is to say for example time series of image stacks or image stacks with different color channels, or five-dimensional, for example time series of image stacks with different color channels.

Preferably, the signature 250 output by the machine learning model 140 is a feature map of a layer of the model. In particular, a last slice before a classification slice of a machine learning model 140 can output the signature 250, and an earlier slice can likewise output the signature 250. According to a further configuration, a plurality of slice outputs from different depths of the machine learning model 140 can also be combined.

In particular, the model can be improved by continuous learning over a plurality of experiments.

According to a further configuration of the first to third embodiments, the images recorded by the microscope 100 can be input into a candidate extraction model in particular as input data. The candidate extraction model outputs a plurality of candidate images, wherein respective sample structures 300 of interest, so-called candidate sample structures, are captured in focus by the microscope 100 in the candidate images. The sample structures 300 of interest can in particular be cell edges, cell organelle, cell nucleus, a cytoskeleton or mitochondria.

In particular, the candidate extraction model is trained over the course of a plurality of experiments in that respective target images selected during the method for repositioning the focus position of the microscope 100 are input into the candidate extraction model. The respective target sample structures are captured in focus by the microscope 100 in the selected target images. The candidate extraction model then learns the recognition of the target sample structures illustrated in focus in the target images.

According to one configuration, the machine learning model 140 can be for example the candidate extraction model. The candidate extraction model is first randomly initialized as described above or pre-trained with a non-specific image data set or with a specific image data set and then continuously learns the recognition of the target sample structures.

In particular, the candidate extraction model can be embodied as a detection model, then the candidate extraction model outputs only the image regions with the sample structures 300 of interest as candidate images. Alternatively, the candidate extraction model can also be implemented as a classifier or semantic segmenting.

According to a further configuration, the method for repositioning the focus position of the microscope 101 comprises inputting the comparison stack into a new focus position determination model and outputting the new focus position by the new focus position determination model.

The new focus position determination model can in particular have been trained based on previously carried out experiments in which the new focus position for holding the target focus position based on a compare stack has been respectively determined over the course of the experiment for outputting the new focus position. A training of the new focus position determination model is in particular a supervised training in which compare stacks and compare focus positions are respectively captured as input data in an annotated data set and the target image and the new focus position are captured as target data. Alternatively, the compare signatures and the compare focus position of the compare stacks can also be used as input data in the annotated data set and the target signatures and the target focus position can be used as target data.

According to a further configuration of the above embodiments, an image recorded for determining the target signature can have been recorded under other recording modalities, called target recording modality, than a recording modality which is used in the experiment currently to be carried out, called experiment recording modality. As described further above, different microscope types, different contrasts or different illuminations, filters or fluorophores can be used. If a different recording modality is then used during the experiment, the embedding of the images recorded in the experiment recording modality into the embedding space corresponding to the second embodiment has to be trained over the course of the experiment in order that the images recorded in the experiment recording modality in the target focus position are also mapped onto the target signature and, correspondingly, the compare images are also suitably embedded into the embedding space.

According to a fourth embodiment, an evaluation device 130 with means for carrying out the method according to the first to the third embodiment is provided.

According to a fifth embodiment, a computer program product is provided which comprises commands which, when the program is executed by a computer, cause the latter to carry out the method according to the first three embodiments described above.

With its sixth embodiment, a repositioning system 1 is provided, comprising the evaluation device 130 according to the fourth embodiment.

The variants and configurations described with reference to the various figures can be combined with one another. The configurations shown and described are purely illustrative and modifications thereof are possible within the scope of the appended claims.

1. A method for repositioning a focus position of an imaging device into a target focus position in a sample over the course of an experiment, comprising:
defining the target focus position,
repositioning a current focus position based on the target focus position, repeatedly comprising:
determining one or more compare signatures based on a current focus position,
determining one or more signature distances in each case between the compare signatures and a target signature based on the target focus position,
adapting the current focus position based on the signature distances,
wherein
a signature is an output of a machine learning model corresponding to a focus position and based on an image of the sample recorded with the focus position, and the target focus position is a focus position in the sample in which a target imaging device captures a target image of the sample and the machine learning model outputs the target signature when the target image is input.

2. The method according to claim 1, wherein the defining of the target focus position comprises:
recording the target image, and
outputting the target signature based on the target image, wherein the target signature comprises an embedding of the target image into an embedding space determined by the machine learning model.

3. The method according to claim 2, wherein recording the target image comprises:
recording one or more images with mutually height-offset focus positions, comprising the target image, and
selecting the target image from the plurality of images.

4. The method according to claim 3, further comprising:
inputting the plurality of images into the machine learning model,
identifying the target image based on the target signature.

5. The method according to claim 3, wherein the selecting of the target image comprises:
inputting the plurality of images into the machine learning model, wherein the machine learning model comprises a candidate extraction model,
outputting a plurality of candidate images by the candidate extraction model such that images from the plurality of images that do not capture candidate sample structures are filtered out by the candidate extraction model, and
selecting the target image from the candidate images, wherein candidate sample structures are captured in the candidate images, and the candidate sample structures are biological structures of interest in the sample, including one or more of:
cell edge,
cell organelle,
cell nucleus,
cytoskeleton, or
mitochondria.

6. The method according to claim 3, wherein the machine learning model has been trained over the course of a plurality of experiments, wherein the machine learning model has been trained based on target images selected in the course of the plurality of experiments for outputting the candidate images, and wherein the candidate extraction model recognizes sample structures represented in the target images with a high image sharpness.

7. The method according to claim 1, wherein the target focus position in the sample is variable over the course of the experiment over time or the target signature is variable over time or both.

8. The method according to claim 1, wherein the target image or the target signature is contained in a sample structure atlas, wherein the sample structure atlas comprises atlas images or atlas signatures for sample structures of interest occurring in a sample of a specific sample type, including biological structures of interest, which represent a change over time of the sample.

9. The method according to claim 8, wherein the sample structure atlas was recorded in one or more previous experiments with a sample of the same sample type.

10. The method according to claim 1, wherein the determining of the compare signature comprises recording a compare stack comprising a plurality of images with compare focus positions being height offset to one another and the determining of the signature distances in each case comprises determining a signature distance between the compare signatures based on the images of the compare stack and the target signature.

11. The method according to claim 10, wherein the recording of a compare stack takes place once a signature distance between the target signature and a compare signature based on an image recorded with the current focus position is greater than a predetermined value.

12. The method according to claim 10, wherein the repositioning of the focus position takes place in a plurality of repositioning rounds, wherein a height offset of the focus positions of the images of the compare stack to one another is reduced in successive repositioning rounds.

13. The method according to claim 1, wherein the adapting of the current focus position comprises determining a new focus position and adapting the current focus position to the new focus position and the determining of the new focus position takes place based on the signature distances between the compare signatures and the target signature.

14. The method according to claim 13, wherein the determining of the new focus position comprises one or more of the following:
selecting the compare focus position corresponding to the compare signature with the smallest signature distance as new focus position, or
calculating the new focus position based on at least one determined signature distance and a correspondence between signature distance and height offset of focus positions.

15. The method according to claim 14, wherein the calculating of the new focus position comprises:
calculating a height offset based on a determined distance transformation, wherein the distance transformation maps a signature distance between two signatures output by the machine learning model to a corresponding height offset of the focus positions of the underlying images, wherein the distance transformation was determined from a sample of the sample type.

16. The method according to claim 1, wherein the machine learning model comprises one or more of the following:
a machine learning model initialized with random weights,
a machine learning model pre-trained with non-specific image data, wherein the machine learning model was trained on the non-specific image data for identifying objects in the image data, and
a machine learning model pre-trained on specific image data, wherein the specific image data comprise image data from a previous experiment with a sample of the same sample type or are image data from a sample structure atlas.

17. The method according to claim 16, wherein a training of the machine learning model comprises one or more of the following training methods:
a supervised learning,
training the machine learning model for classifying image data,
training an embedding model comprised by the machine learning model for embedding into an embedding space, wherein the embedding model is trained to map embeddings of images with height-offset focus positions into the embedding space such that a distance of the images with height-offset focus positions corresponds to a distance of the embeddings of the images in the embedding space, and
training a metric model comprised by the machine learning model, wherein the metric model is trained to respectively assign a signature distance to two input signatures such that the height offset of the height-offset focus positions is mapped to a corresponding signature distance.

18. The method according to claim 17, wherein the training of the machine learning model comprises a training of a main task and a training of an auxiliary task, wherein the main task comprises the outputting of the signature and the auxiliary task comprises one or more of the following auxiliary tasks:
classifying the image data, including classifying the image data on the basis of sample structures contained in the image data,
semantic segmenting of the image data,
classifying the image data, wherein the image data are divided at least into image data with sample structures of interest and image data without sample structures of interest,
carrying out a transformation, and
a pseudo-task, in which a random noise is added to calculated gradients during the training.

19. The method according to claim 1, further comprising:
selecting the machine learning model from a series of pre-trained machine learning models, including automatically selecting the machine learning model based on a first image recorded by the sample, an overview image and/or context information.

20. A method for training a machine learning model for outputting a signature, wherein the signature is suitable for being used in a method for repositioning a focus position of an imaging device according to claim 1.

21. An evaluation device for repositioning a focus position of an imaging device, comprising means for carrying out the method according to claim 1.

22. An evaluation device for training a machine learning model according to the method according to claim 20.

23. A repositioning system, comprising the evaluation device according to claim 21, and comprising a microscope.

24. A computer program product, comprising commands which, when the program is executed by a computer, cause the computer to carry out the method according to claim 1.
